(19) United States
(12) Patent Application Publication
(10) Pub. No.: US 2021/0228823 A1
(43) Pub. Date: Jul. 29, 2021

(54) Urinary Catheter Suited for Hyperbaric Oxygen Therapy for Prostatitis

(71) Applicants: Mohammed Muddassir Mohiuddin, Frederick, MD (US); Aabid Mohammed Mohiuddin, Frederick, MD (US)

(72) Inventors: Mohammed Muddassir Mohiuddin, Frederick, MD (US); Aabid Mohammed Mohiuddin, Frederick, MD (US)

(21) Appl. No.: 17/151,598

(22) Filed: Jan. 18, 2021

Related U.S. Application Data
(60) Provisional application No. 62/966,109, filed on Jan. 27, 2020.

(51) Int. Cl. A61M 13/00; A61M 25/00; A61M 25/10

(52) U.S. Cl. CPC A61M 13/003; A61M 25/0017; A61M 25/10; A61G 10/026; A61M 2210/1085; A61M 2202/0208; A61M 25/007

(57) ABSTRACT

The present invention is a urinary catheter with media injection channel suited for prostatitis treatment and therapy. The urinary catheter with media injection channel contains a catheter body, an injection channel, and at least one injection aperture. The catheter body contains a bladder opening, a balloon, and a utility port. The bladder opening and the utility port are positioned terminally opposite to each other along the catheter body. The balloon is positioned between the bladder opening and the utility port. At least one injection aperture is positioned adjacent to the balloon, opposite to the bladder opening. At least one injection aperture is distributed about the catheter body. The injection channel traverses from the utility port to the at least one injection aperture.

The current application claims a priority to the U.S. Provisional Patent application Ser. No. 62/966,109 filed on Jan. 27, 2020.

### FIELD OF THE INVENTION

The present invention relates to catheters, specifically catheters with an additional channel for hyperbaric oxygen therapy along a patient's prostate.

### BACKGROUND OF THE INVENTION

It is believed that chronic prostatitis is one of the most common reasons why men visit urologists, even being characterized as the condition responsible for more outpatient visits than benign prostatic hypelplasia (“BPH”) or prostate cancer. At least one report states that 35-50% of men will be affected by prostatitis at some time in their life. The treatments conventionally used to treat this condition have been generally problematic; most of the treatments have provided little hope that the condition could be predictably treated in a manner which could successfully alleviate the pain experienced by a large percentage of these individuals. Indeed, prostatitis has been termed “a waste basket of clinical ignorance” because of the lack of knowledge about the basic epidemiology of the disease and also the diagnosis and treatments available for same. Unlike BPH, which occurs primarily in older men, prostatitis can occur in both younger (men in age groups of 18-50 (or younger)) and older men (over the age of 50), with the median reported patient age at about 40 years of age.

Various treatment protocols have been used to attempt to treat prostatitis. While category I may be managed successfully with wide spectrum antibiotics, patients having category II and III prostatitis have had lesser degrees of response success when treated with antibiotics. Other treatment regimens include other drugs such as alpha-blocker therapy (for obstructive voiding), and anti-inflammatory agents. In addition, or alternatively, the physician may suggest lifestyle changes such as diet (such as the reduction of the intake of caffeine), exercise, sexual activity, and/or supportive psychotherapy or “coping mechanisms”. In view of the above, there remains a need to provide improved and/or alternative treatments for chronic prostatitis. In recent studies, it is shown that hyperbaric oxygen therapy can effectively inhibit the growth of tumor cells. In addition, the injection of pure oxygen to the prostate will help stop bleeding for the cancerous prostate. Therefore, it is an object of the invention to provide a means of integrating hyperbaric oxygen therapy to both treat acute and chronic prostatitis.

### SUMMARY OF THE INVENTION

The present invention is a urinary catheter with media injection channel suited for prostatitis treatment and therapy. The urinary catheter with media injection channel comprises a catheter body, an injection channel, and at least one injection aperture. In the preferred embodiment of the present invention, the urinary catheter may take the form of a foley catheter with an additional channel for oxygen injection to facilitate hyperbaric oxygen therapy along the patients prostate. The catheter body comprises a bladder opening, a balloon, and a utility port.

The bladder opening and the utility port are positioned terminally opposite to each other along the catheter body. In the preferred embodiment of the present invention, the bladder opening is the patient input end of the urinary catheter that allows a user to install the urinary catheter to the patient's bladder. The utility port of the urinary catheter serves as the region for urine extraction, oxygen delivery, or balloon inflation input. The balloon is positioned between the bladder opening and the utility port. At least one injection aperture is positioned adjacent to the balloon, opposite to the bladder opening. At least one injection aperture is distributed about the catheter body. The injection channel traverses from the utility port to the at least one injection aperture. The balloon, when inflated, stops the urinary catheter from coming out the patient's bladder. The balloon will also be used to block incoming oxygen from going into the patient's bladder. The injection channel serves as the oxygen injection channel that allows the user to inject pure oxygen directly to the prostate along the at least one injection aperture, facilitating hyperbaric oxygen therapy administration to the affected prostate.

### BRIEF DESCRIPTION OF THE DRAWINGS

FIG. 1 is a top perspective view of the present invention.

FIG. 2 is a top view of the present invention with cutting lines A-A.

FIG. 3 is sectional view of the present invention taken along cutting lines A-A in FIG. 2.

FIG. 4 is a top view of the present invention with cutting lines B-B.

FIG. 5 is sectional view of the present invention taken along cutting lines B-B in FIG. 4.

FIG. 6 is a top view of the present invention with cutting lines C-C.

FIG. 7 is sectional view of the present invention taken along cutting lines C-C in FIG. 6.

FIG. 8 is a top view of the present invention with cutting lines D-D.

FIG. 9 is sectional view of the present invention taken along cutting lines D-D in FIG. 8.

FIG. 10 is a diagram view of a utility port used in the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

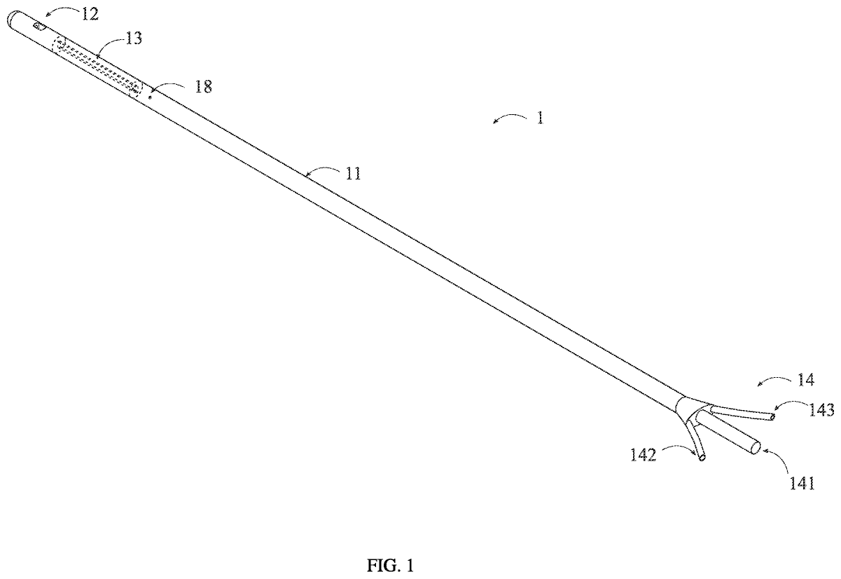

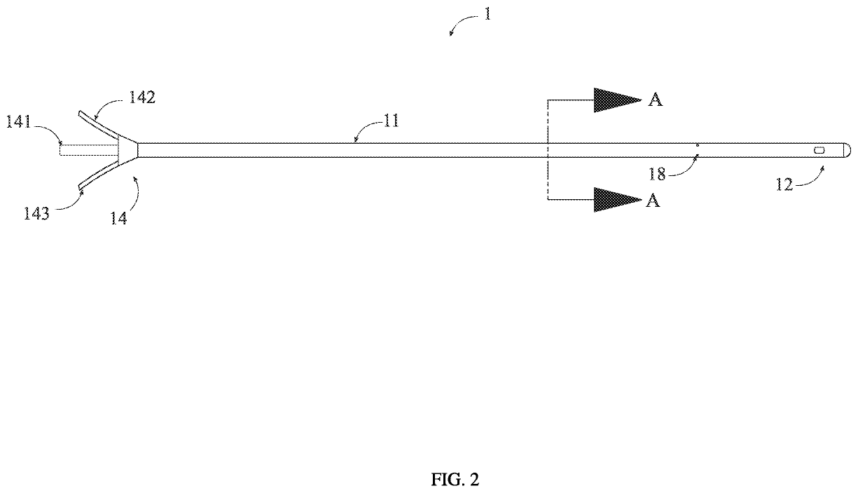

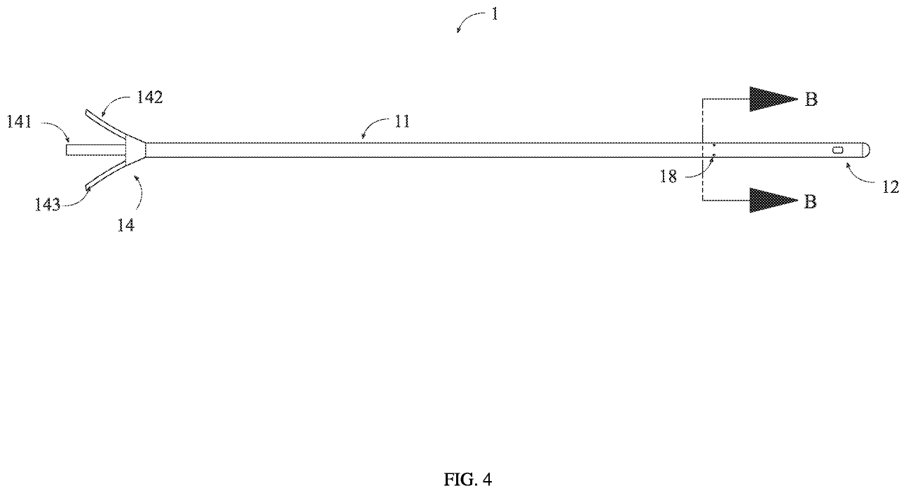

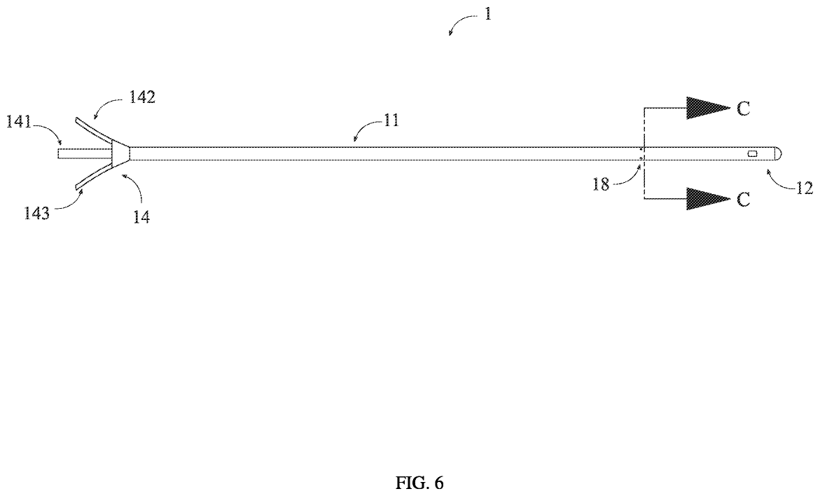

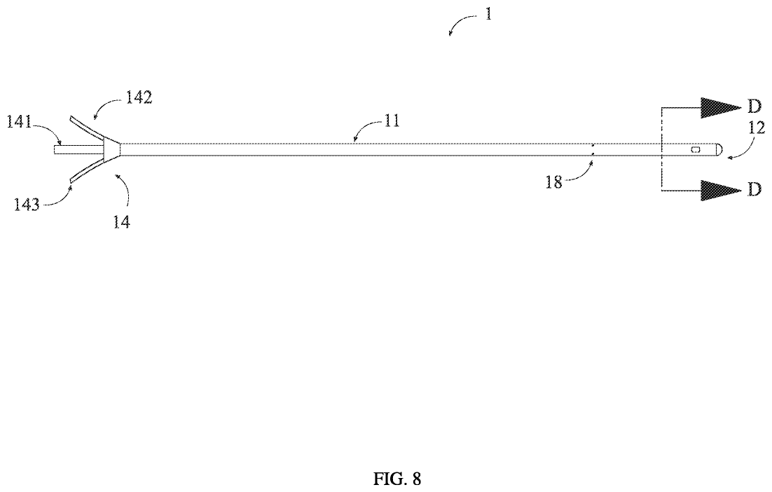

In reference to FIGS. 1-2, 4, 6, and 8, the present invention is a urinary catheter with media injection channel 1 suited for prostatitis treatment and therapy. The urinary catheter with media injection channel 1 comprises a catheter body 11, an injection channel 16, and at least one injection aperture 18. In the preferred embodiment of the present invention, the urinary catheter with media injection channel 1 may take the form of a foley catheter with an additional channel for oxygen injection to facilitate hyperbaric oxygen therapy along the patients prostate. In the preferred embodiment of the present invention, the urinary catheter with media injection channel 1 can be made out of any suitable material, suitable for surgical and/or therapeutic implications such as, but not limited to medical grade silicone, rubber, polymers, or any other suitable material. In the preferred embodiment of the present invention, the urinary catheter with media injection channel 1 can be scaled to any size, configuration, style to suitable fit a patient. The catheter body 11 comprises a bladder opening 12, a balloon 13, and a utility port 14.

The bladder opening 12 and the utility port 14 are positioned terminally opposite to each other along the catheter body 11, as shown in FIGS. 1-2, 4, 6, and 8. In the preferred embodiment of the present invention, the bladder opening 12 is the patient input end of the urinary catheter with media injection channel 1 that allows a user to install the urinary catheter with media injection channel 1 to the patient's bladder. The utility port 14 of the urinary catheter with media injection channel 1 serves as the region for urine extraction, oxygen delivery, or balloon 13 inflation input. The balloon 13 is positioned between the bladder opening 12 and the utility port 14. In reference to FIGS. 1-6 and 8, at least one injection aperture 18 is positioned adjacent to the balloon 13, opposite to the bladder opening 12. At least one injection aperture 18 is distributed about the catheter body 11. The injection channel 16 traverses from the utility port 14 to the at least one injection aperture 18. The balloon 13, when inflated, stops the urinary catheter from coming out the patient's bladder. The balloon 13 will also be used to block incoming oxygen from going into the patient's bladder. The injection channel 16 serves as the oxygen injection channel 16 that allows the user to inject pure oxygen directly to the prostate along the at least one injection aperture 18, facilitating hyperbaric oxygen therapy administration to the affected prostate.

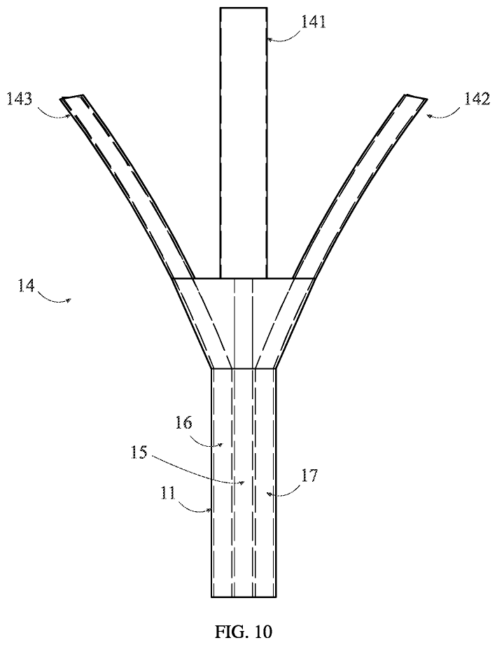

The utility port 14 comprises an injection port 143, as shown in FIGS. 1-2, 4, 6, 8, and 10. The injection port 143 is connected adjacent to the injection channel 16, opposite to the at least one injection aperture 18. In the preferred embodiment of the present invention, the injection port 143 may take the form of any suitable pneumatic connector inlet that facilitates the connection of an oxygen injection apparatus that dispenses pure oxygen along the injection port 143.

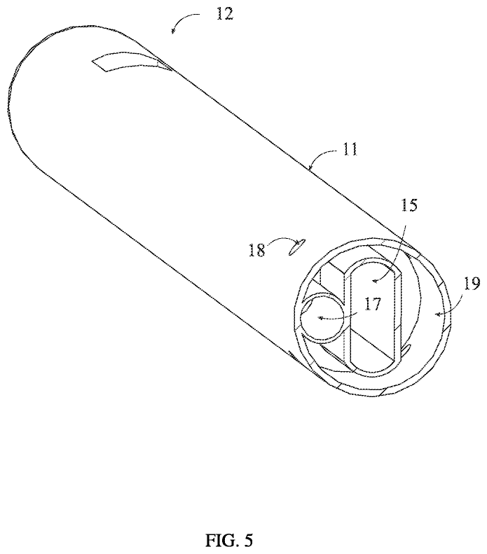

The urinary catheter with media injection channel 1 further comprises an injection cavity 19, as shown in FIG. 5. The injection cavity 19 is positioned within the catheter body 11. The injection cavity 19 is positioned adjacent to the balloon 13, opposite to the bladder opening 12. The injection channel 16 traverses from the utility port 14 to the injection cavity 19. At least one injection aperture 18 traverses from the catheter body 11 to the injection cavity 19. In the preferred embodiment of the present invention, the injection cavity 19 may take the form of an air pocket within the catheter body 11 that fills with pure oxygen gas such that the pure oxygen gas distributes along the at least one injection aperture 18.

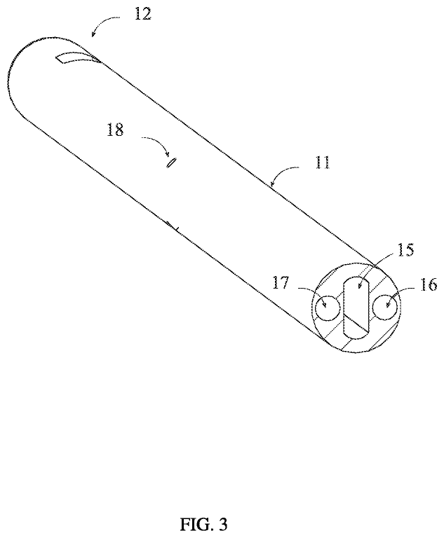

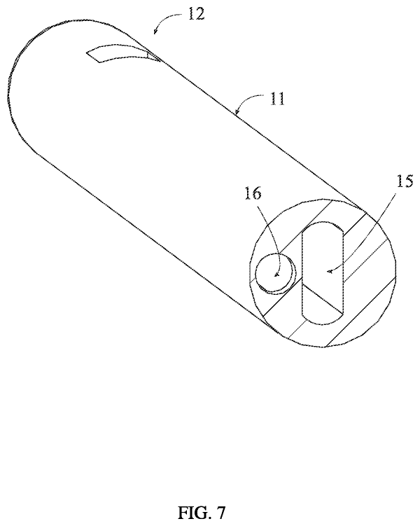

The utility port 14 comprises a waste port 141, as shown in FIGS. 1-2, 4, 6, 8, and 10. The urinary catheter with media injection channel 1 further comprises a waste channel 15, as shown in FIGS. 3, 5, 7, and 9. The waste port 141 is positioned adjacent to the utility port 14, opposite to the bladder opening 12. The waste channel 15 traverses from the waste port 141 to the bladder opening 12. In the preferred embodiment of the present invention, the waste channel 15 serves as the urine extraction channel that dispenses waste urine out of the bladder opening 12. The waste port 141 facilitates the connection of a waste disposal receptacle to maintain a sterile and sanitized means of waste extraction.

The utility port 14 comprises an inflation port 142, as shown in FIGS. 1-2, 4, 6, 8, and 10. The urinary catheter with media injection channel 1 further comprises an inflation channel 17, as shown in FIGS. 3, 5, and 7. The inflation port 142 is positioned adjacent to the utility port 14, opposite to the bladder opening 12. The inflation channel 17 traverses from the inflation port 142 to the balloon 13. In the preferred embodiment of the present invention, the inflation channel 17 serves as the balloon inflation channel 17 that allows the user to inflate the balloon 13 to stop the urinary catheter with media injection channel 1 from coming out of the patient's bladder. The inflation port 142 facilitates the connection of any suitable balloon 13 inflation device.

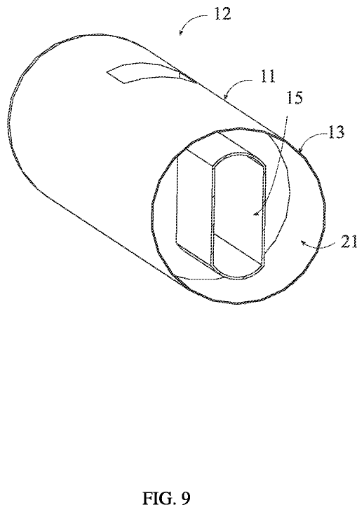

The urinary catheter with media injection channel 1 further comprises a balloon cavity 21, as shown in FIG. 9. The balloon cavity 21 is positioned within the balloon 13. The inflation channel 17 traverses from the inflation port 142 to the balloon cavity 21. In the preferred embodiment of the present invention, the balloon cavity 21 may take the form of a sealed air pocket that is pneumatically connected to the inflation channel 17. When inflated, the section of the catheter body 11 that resides along the balloon cavity 21 expands, forming the balloon 13.

Although the invention has been explained in relation to its preferred embodiment, it is to be understood that many other possible modifications and variations can be made without departing from the spirit and scope of the invention as hereinafter claimed.

What is claimed is:

1. A urinary catheter with media injection channel comprising:
a catheter body;
an injection channel;
at least one injection aperture;
the catheter body comprising a bladder opening, a balloon, and a utility port;
the bladder opening and the utility port being positioned terminally opposite to each other along the catheter body;
the balloon being positioned between the bladder opening and the utility port;
at least one injection aperture being positioned adjacent to the balloon, opposite to the bladder opening;
at least one injection aperture being distributed about the catheter body;
the injection channel traversing from the utility port to the at least one injection aperture.

2. The urinary catheter with media injection channel, as claimed in claim 1, comprising:
the utility port comprising an injection port; and
the injection port being connected adjacent to the injection channel, opposite to the at least one injection aperture.

3. The urinary catheter with media injection channel, as claimed in claim 1, comprising:
an injection cavity;
the injection cavity being positioned within the catheter body;
the injection cavity being positioned adjacent to the balloon, opposite to the bladder opening;
the injection channel traversing from the utility port to the injection cavity; and
at least one injection aperture traversing from the catheter body to the injection cavity.

4. The urinary catheter with media injection channel, as claimed in claim 1, comprising:
the utility port comprising a waste port;
a waste channel;
the waste port being positioned adjacent to the utility port, opposite to the bladder opening; and
the waste channel traversing from the waste port to the bladder opening.

5. The urinary catheter with media injection channel, as claimed in claim 1, comprising:
the utility port comprising an inflation port;
an inflation channel;
the inflation port being positioned adjacent to the utility port, opposite to the bladder opening; and
the inflation channel traversing from the inflation port to the balloon.

6. The urinary catheter with media injection channel, as claimed in claim 5, comprising:
a balloon cavity;
the balloon cavity being positioned within the balloon; and
the inflation channel traversing from the inflation port to the balloon cavity.
